(19) United States
(12) Patent Application Publication
(10) Pub. No.: US 2019/0216575 A1
(43) Pub. Date: Jul. 18, 2019

(54) SYSTEM AND METHOD FOR CO-REGISTERING A STEREOTACTIC FRAME AND A FIDUCIAL

(71) Applicant: Alpha Omega Neuro Technologies Ltd., Nazareth (IL)

(72) Inventor: Maroun Farah, Nazareth (IL)

(73) Assignee: Alpha Omega Neuro Technologies Ltd., Nazareth (IL)

(21) Appl. No.: 16/365,856

(22) Filed: Mar. 27, 2019

Related U.S. Application Data
(63) Continuation of application No. 14/935,341, filed on Nov. 6, 2015, now abandoned.
(60) Provisional application No. 62/075,951, filed on Nov. 6, 2014.

(51) Int. Cl. A61B 90/00

(52) U.S. Cl. CPC A61B 90/39; A61N 1/3605; A61B 2090/3983

(57) ABSTRACT

The disclosure relates to methods, systems and devices for positioning a stereotactic frame within a desired surgical site, and more particularly to stereotactic systems and methods of co-registration of stereotactic frames with imbedded fiducial markers.

### RELATED APPLICATIONS

This application is a continuation of U.S. patent application Ser. No. 14/935,341 filed on Nov. 6, 2015, which claims the benefits of priority from U.S. Provisional Patent Application No. 62/075,951, filed on Nov. 6, 2014, the contents of which are incorporated herein by reference in their entirety.

### FIELD AND BACKGROUND OF THE INVENTION

The disclosure is directed to methods, systems and devices for positioning a stereotactic frame within a desired surgical site, and more particularly to stereotactic systems and methods of co-registration of stereotactic frames with imbedded fiducial markers.

Deep brain stimulation (DBS) is a surgical procedure involving the implantation of a medical device called a macroelectrode (also referred to as a “lead”, “brain pacemaker”, “electrode” or “chronic electrode”), which sends electrical impulses to specific parts of the brain. DBS in select brain regions has provided noticeable therapeutic benefits for otherwise treatment-resistant movement and affective disorders such as chronic pain, Parkinson's disease, tremor, dystonia and depression. At present, the procedure is used only for patients whose symptoms cannot be adequately controlled with medications. DBS directly changes brain activity in a controlled manner, and its effects are reversible (unlike those of lesioning techniques). DBS uses the surgically imbedded, battery-operated medical neurostimulator to deliver electrical stimulation to targeted areas in the brain that control movement, blocking the abnormal nerve signals that cause tremor and PD symptoms.

The common method of performing a brain surgery, or deep brain stimulation on a patient involves the following steps: Before the surgery the patient undergoes an MRI scan in order to identify the targeted surgical site and entry point in the skull; a base is attached and fixed in a rigid manner to the patients head; a scanning frame is attached to the base, whereas the scanning frame usually includes several rods, which are made of material that is detectable using imaging modalities but does not cause any distortion thereto, the rods spatial orientation is in a fixed relation with respect to the frame base; once the scan is completed, the relative position of the frame base is compared to the patient imaging markers; a surgical frame system is attached to the frame base; since the relation between the frame and the base is known, calculation of relation between the frame and the targeted surgical site can be performed and the frame can be adjusted so that the surgical tool reaches the targeted surgical site, whereas in some of the frames, the skull entry angle can be adjusted

DBS systems typically consist of several components, such as the macroelectrode, the extension, the neurostimulator, and a stereotactic frame used to accurately guide the electrode to the target area in the brain. The macroelectrode—a thin, insulated wire—is inserted through a small opening in the skull and imbedded in the brain. The tip of the electrode is positioned within the targeted brain area.

Once the targeted surgical site is identified, a reference external structure, such as the stereotactic frame, has to be positioned in a fixed relation with respect to the patient body in order to enable establishing a relationship between the reference structure and the targeted surgical site.

In some cases, the reference structure is an external component that is not attached to the body of a patient, rather the geometric relation between the imaging markers and the reference structure is performed through various coupling techniques, such as scanning the imaging markers and the reference structure by another system. Alternatively, a system of fiducials attached to the patient body can be used, whereas the position of the fiducials is available using the imaging techniques and the reference structure system can co-register to these positions.

Once the system is in place, electrical impulses are sent from the neurostimulator up along the extension wire and the lead and into the brain. These impulses interfere with and block the electrical signals that cause the undesired symptoms. The person has the possibility to turn the DBS off if required.

One of the frequently used reference structures is a stereotactic frame system that includes a base, a scanning frame and two arcs, such as CRW from Redionics and Leksell frame from Integra that are used for cranial applications. Scanning frame was used to determine the geometrical relations of various anatomical markers and subsequently replaced by a surgical frame.

Two coordinate systems are used in order to identify the targeted surgical site. The first coordinate system belongs to the imaging markers, including the targeted surgical site within the patient body, the second coordinate system belongs to the frame. These two coordinate systems are co-registered in order to enable navigation towards the targeted surgical site using the external frame coordinate system.

Different guiding methods use different types of fiducials. One common type of fiducials involves a screw that is threaded into the skull while keeping a portion of the fiducial exposed above the skin of the patient. The fiducials are commonly detected using imaging techniques as well as using an external sensing system and subsequent co-registration between the imaging markers and external system readings.

Accordingly, accurate and fast co-registration methods and systems are needed to facilitate aligning the imaging markers.

### SUMMARY OF THE INVENTION

Provided herein are embodiments of stereotactic surgical frames, fiducial insertion devices and electrode insertion systems.

In an embodiment, provided herein is a fiducial threading device comprising: a housing having a longitudinal axis an apical end and a basal end, with a bore extending axially; a cannula, operably coupled to the basal end of the housing; a rod, having an apical end coupled to a knob and a basal end coupled to a fiducial; and the fiducial, having an apical end configured to releasably engage the basal end of the rod, and a basal end configured to penetrate and engage a bone tissue of a subject.

In yet another embodiment, provided herein is a stereotactic frame engagement system comprising: a frame pod having a lower end; and a fiducial having an upper end configured for movable point-to-point engagement with said lower end of said frame pod.

In another embodiment, provided herein is a system for co-registering a stereotactic surgical frame with imbedded fiducials, comprising: a stereotactic surgical frame at least three frame pods having edges coupled to the stereotactic surgical frame, configured to position the frame in a predetermined plane; a sensor array operably coupled to the stereotactic surgical frame, configured to communicate with and detect the position of a plurality of fiducial imbedded within a patient body organ; and the plurality of fiducials, imbedded within a patient's body organ, configured to communicate with the sensor array.

In yet another embodiment, provided herein is a stereotactic surgical frame for facilitating insertion of a surgical tool into a surgical site within a patient body, comprising: a convex domed portion having an open circumferential basal lip, the convex dome defining an aperture at its apex, the aperture configured to receive and engage a spherical cap portion ; the spherical cap portion, being movable with respect to said spherical domed portion and concentric therewith, movably coupled to the aperture of the convex domed portion; and at least three support rods, having an upper end operably coupled to the open circumferential basal lip.

In yet another embodiment, provided herein is a kit comprising: a plurality of any of the fiducials provided herein; any of the fiducial insertion devices provided herein; any of the stereotactic surgical frames provided herein; optionally an electrode; optionally packaging; and optionally instructions.

### BRIEF DESCRIPTION OF THE SEVERAL VIEWS OF THE DRAWINGS

The patent or application file contains at least one drawing executed in color. Copies of this patent or patent application publication with color drawings will be provided by the Office upon request and payment of the necessary fee.

The features of the stereotactic surgical frames, fiducial insertion devices and electrode insertion systems described herein, will become apparent from the following detailed description when read in conjunction with the drawings, which are exemplary, not limiting, and wherein like elements are numbered alike in several figures and in which:

FIG. 1A, is a simplified illustration of an embodiment of the fiducial insertion device, with an enlarged portion A illustrated in FIG. 1B;

FIG. 2A-2C are a simplified illustration of the use of an embodiment of the fiducial insertion device for imbedding a fiducial, with the final fiducial positioning illustrated in FIG. 2D.

FIG. 3A-3E are simplified illustrations of an embodiment of fiducial assembly, with, FIG. 3E is taken along lines C-C of FIG. 3D;

FIG. 4A is a simplified illustration of another embodiment of the fiducial insertion device shown in FIGS. 3A-3E into a body of a patient, wherein the fiducial is positioned within a needle, with X-Z cross section thereof illustrated in FIG. 4B;

FIG. 5 illustrates another embodiment of the fiducial insertion device shown in FIGS. 3A-3E into a body of a patient, wherein the fiducial forms part of the needle;

FIGS. 6A-6C illustrate various stages of imbedding a fiducial into a skull of a patient using an embodiment of the fiducial insertion device shown in FIG. 4 (same stages can be achieved using the fiducial insertion device shown in FIG. 5;

FIG. 7 is a simplified illustration of an embodiment of a stereotactic frame pod assembly;

FIG. 8A illustrates an embodiment of the upper portion of a fiducial used in conjunction with the frame pod of FIG. 7, with FIGS. 8B-8C illustrating possible configurations thereof and FIG. 8D, illustrating a X-Z cross section of the configuration of FIG. 8C, movably coupled to and engaged therein;

FIGS. 9A-9C illustrate another embodiment of the engagement configuration between the frame pod of FIG. 7 and the upper end of the fiducial of FIG. 8A;

FIG. 10A illustrates a bell configuration of the upper portion of a fiducial, with a Z-X cross section thereof illustrated in FIG. 10B, while the lower portion of the frame pod coupling process with the fiducial of FIG. 10A illustrated in FIGS. 10C-10D, and a Z-X cross section of the coupled configuration illustrated in FIG. 10E;

FIGS. 11A-11C are simplified illustrations of another embodiment of fiducial imbedding assembly, and a pod engagement configuration with, FIG. 11C illustrating a Z-X cross section of FIG. 11B;

FIG. 12 illustrates a stereotactic frame undergoing co-registration process with an imaging coordinate system;

FIG. 13 illustrates another embodiment of a stereotactic frame undergoing co-registration process with an imaging coordinate system with a different frame pod configuration undergoing adjustment;

FIG. 14 Illustrates the spatial arrangement of the surgical tool or probe and the stereotactic frame operable where the tip of the probe is positioned at the center of the arc.

FIG. 15, illustrates a schematic representation of a stereotactic frame operable in a spherical coordinate system;

FIG. 16, illustrates the spatial arrangement of the surgical tool or probe and the stereotactic frame operable in a spherical coordinate system; and

FIG. 17, illustrates the spatial arrangement of the surgical tool or probe and the stereotactic frame operable in a spherical coordinate system with the vertical maneuverability of an electrode within the frame.

While the disclosure is amenable to various modifications and alternative forms, specifics thereof have been shown by way of example in the drawings and will be further described in detail herein below. It should be understood, however, that the intention is not to limit the disclosure to the particular embodiments described. On the contrary, the intention is to cover all modifications, equivalents, and alternatives.

### DESCRIPTION OF SPECIFIC EMBODIMENTS OF THE INVENTION

The disclosure relates in one embodiment to stereotactic systems and methods of co-registration of stereotactic frames with inserted fiducial markers.

The disclosure provides for a fiducial threading device comprising: a housing having a longitudinal axis an apical end and a basal end, with a bore extending axially; a (potentially sharpened) cannula, operably coupled to the basal end of the housing; a rod, having an apical end coupled to a knob on top of the rod and a basal end coupled to a fiducial; and the fiducial, having an apical end configured to be releasably engaged by the basal end of the rod, and the basal end of the fiducial, configured to penetrate, engage and become imbedded in a bone tissue of a subject, in other words, be substantially surrounded by the bone tissue and does not protrude above the bone. The devices for imbedding fiducials used in the systems and kits provided, are configured to imbed a fiducial without the need to make any incisions or for that matter, suture the site of the imbedded fiducials, thus reducing trauma to the patient, accelerating healing time. This is achieved by, for example, attaching a miniaturized fiducial at the end of a needle having disconnect means configured to release the fiducial from a plunger rod once imbedded within the skull without protruding above the bone. The disconnect means can be, for example a failure point, a shear zone, reverse threading to the fiducials and other similar means,

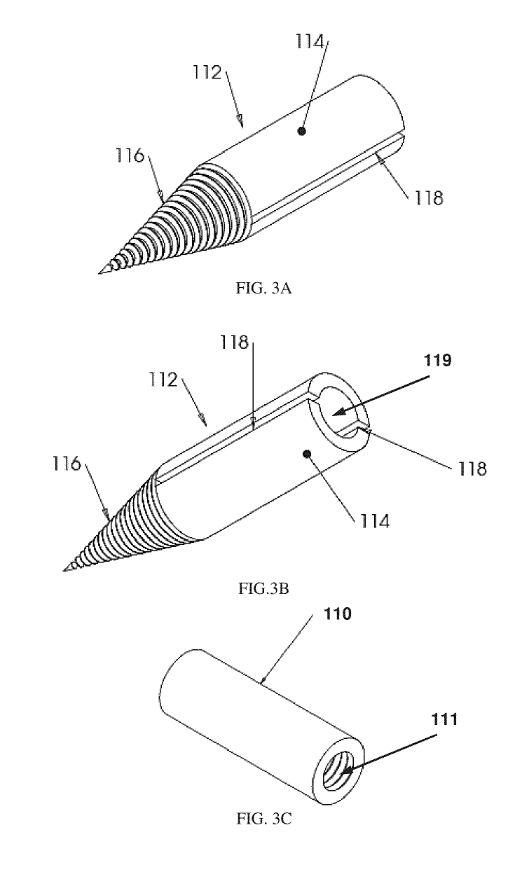

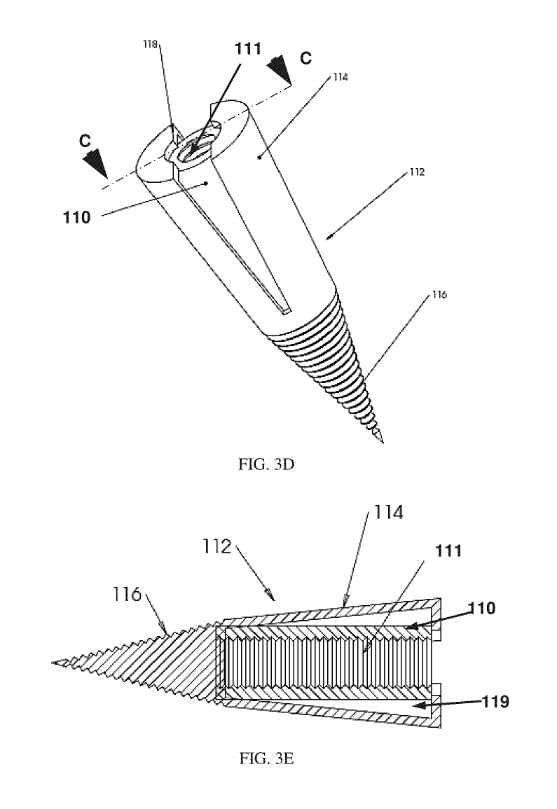

The fiducials used in conjunction with the stereotactic surgical systems and methods of co-registration of stereotactic frames with imbedded fiducial markers described herein can further comprise a conical threaded basal end coupled to an apically open cylinder having a floor, a hollow body with walls rising from the floor and an apical end defining a ceiling having an aperture therein wherein the diameter of the aperture in the ceiling that is smaller than the diameter of the hollow body defined within the cylindrical walls (see e.g., FIGS. 3A-3E), and wherein the cylinder wall defines a couple of diametrically opposed axial slits longitudinally extending substantially along the cylinder walls. The fiducial can further have a cylindrical coupling member or a peg having an internally threaded bore therein, the cylindrical coupling member configured to be accommodated (in other words, fit within with only a small amount of space) in the hollow body of the apically open cylinder of the fiducial, the internally threaded bore configured to rotatably (in other words screw into) and releasably couple to the rod (which can have a basal end with complimentary external threading) and has a threading direction opposite (e.g., counter clockwise) to the threading direction (e.g., clockwise) of the conical threaded basal end of the fiducial. The threading on the conical end of the fiducial can be configured to “bite” into the bone and have for example, sharpened edges. Moreover, the fiducials can also be formed of a biodegradable material and or be radio-opaque material configured to allow the fiducial to be detected during an imaging procedure, for example CT, MRI and the like.

Moreover, the cylindrical coupling member can be used as a receiving element for other members, that when coupled to the cylindrical member, will form a fiducial assembly that can be configured to provide co-registration with a stereotactic surgical frame, as point-to-point coupling site(s).

In another embodiment, provided herein is a stereotactic frame engagement system comprising: a frame pod having a lower end; and a fiducial having an upper end configured for movable point-to-point engagement with said lower end of said frame pod. The frame pod con be an assembly comprised of various components, for example, the support can be a conical member having a narrow lower end configured to movably couple to a fiducial and a wider upper portion configured to give support to the stereotactic frame. Other components can be, for example a transverse coupler bar, configured to provide coupling means to a fixation means (e.g., a nail, a screw, a boss or the like), and fixation means. A pod can comprise all some or more than these components.

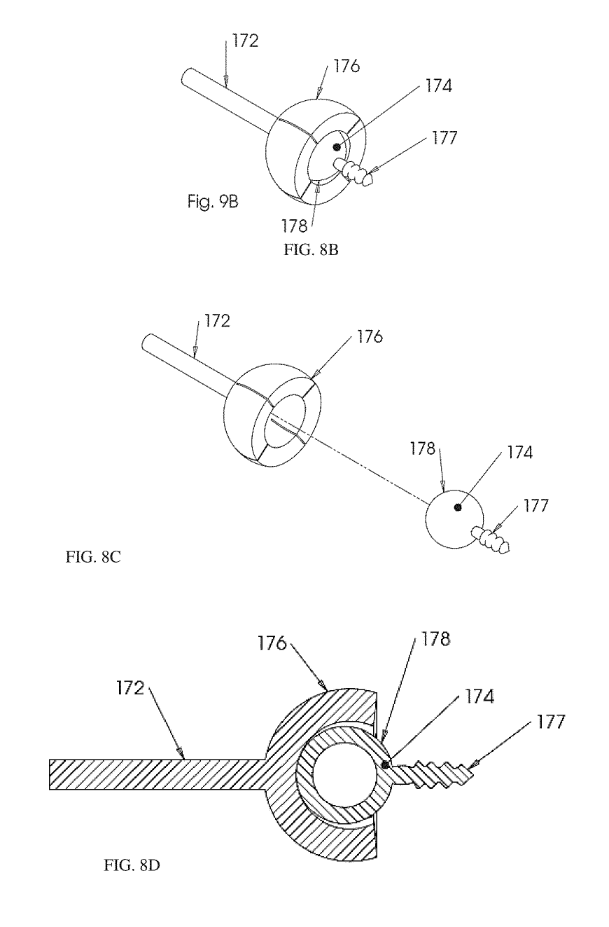

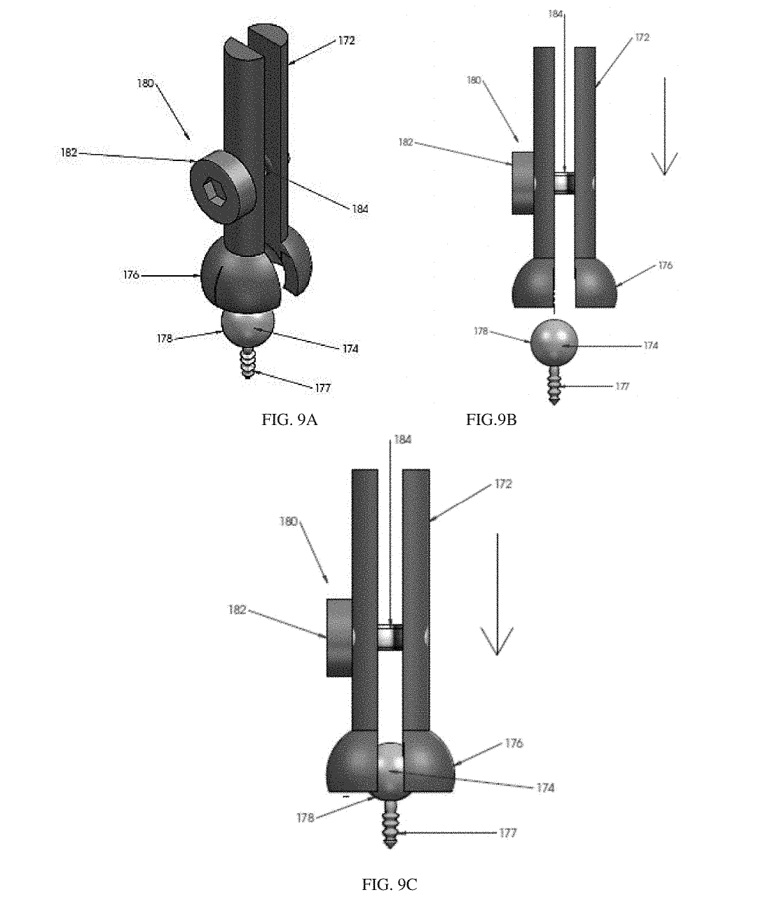

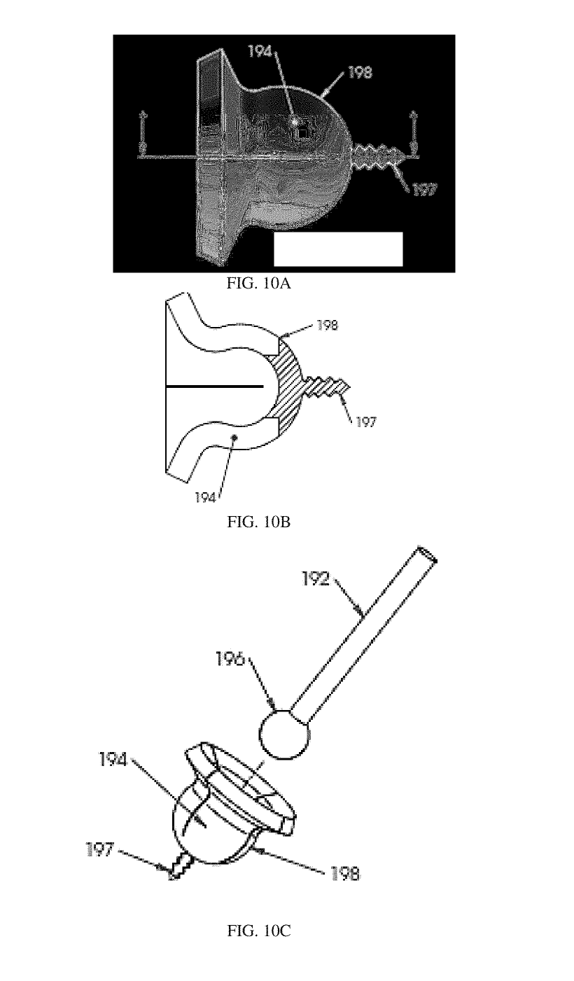

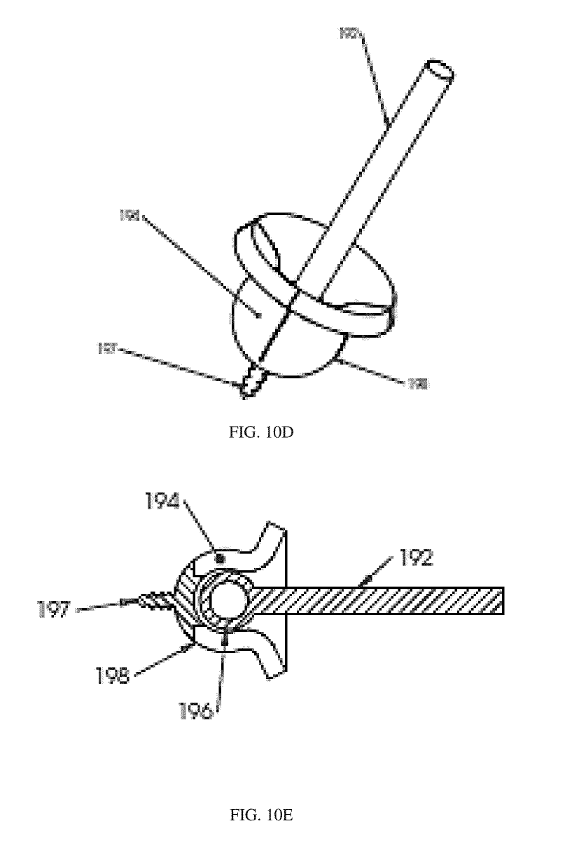

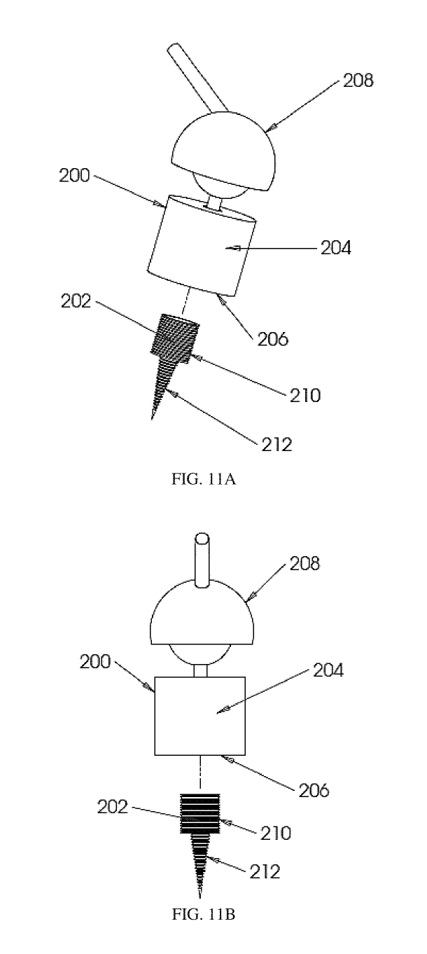

The upper end of the fiducial used in conjunction with the stereotactic systems and methods of co-registration of stereotactic frames with imbedded fiducial markers described herein can be spherical and the lower end of the frame pod can be a semi-spherical concave receiving element having a general bell shape, configured to accommodate the upper (e.g., spherical) end of the fiducial thus providing a rotatable coupling configuration (see e.g., FIGS. 8A-9C). Likewise, the upper end of the fiducial(s) can be a semi-spherical concave receiving element (or bell shaped) configured to accommodate the lower end of the frame pod (or portion thereof) and the lower end of the frame pod can be a sphere (see e.g., FIGS. 10A-10E). Moreover, the upper end of the fiducial can have a cylindrical member with an external threading and the lower end of the frame pod (or a portion thereof) can have a cylindrical member with a bore therethrough having internal threading complimentary to that of the fiducial's. Other engagement configurations can be whereby the upper end of the fiducial can be telescopically coupled to the lower end of said frame pod, and be selectively slidably adjustable.

The term “coupled”, including its various forms such as “operably coupled”, “coupling” or “coupleable”, refers to and comprises any direct or indirect, structural coupling, connection or attachment, or adaptation or capability for such a direct or indirect structural or operational coupling, connection or attachment, including integrally formed components and components which are coupled via or through another component or by the forming process (e.g., an electromagnetic field). Indirect coupling may involve coupling through an intermediary member or adhesive, or abutting and otherwise resting against, whether frictionally (e.g., against a housing) or by separate means without any physical connection.

In certain embodiments, while the bottom portion of the fiducial can be a screw configured to mate with and couple the upper end (either a ball or a snap-on bell shaped coupling), such that while the fiducial marker is below the patient's skin, the upper end can be positioned above the skin.

Further, provided herein is a system for co-registering a stereotactic surgical frame with imbedded fiducials, comprising: a stereotactic surgical frame with at least three frame pods having edges (e.g., at the top of a portion of the frame pods) coupled to the stereotactic surgical frame. The pods (or frame pod assemblies) can be configured to position the frame in a predetermined plane (e.g., above the patient's skull, relative to an external object). A sensor array can be operably coupled to the stereotactic surgical frame, configured to communicate with and detect the position of a plurality of fiducial imbedded within a patient body organ; and the plurality of fiducials, imbedded within a patient's body organ, configured to communicate with the sensor array.

The term “communicate” (and its derivatives e.g., a first component “communicates with” or “is in communication with” a second component) and grammatical variations thereof are used to indicate a structural, functional, mechanical, electrical, or optical relationship, or any combination thereof, between two or more components or elements, for example, appropriate sensors in the sensor array). As such, the fact that one component is said to communicate with a second component is not intended to exclude the possibility that additional components can be present between, and/or operatively associated or engaged with, the first and second components. Furthermore, the term “electronic communication” that can be used to describe the communication between the fiducials and the sensor array in an embodiment, means that one or more components of the sensor array and or fiducial(s) being in electronic communication with sensors in the sensor array and that are described herein are in wired or wireless communication or internet communication so that electronic signals and information can be exchanged between the components.

For example, the sensors' array used in conjunction with the stereotactic systems and methods of co-registration of stereotactic frames with imbedded fiducial markers described herein can comprise transceivers located at a predetermined geometric location with respect to the edges of said frame pods. Those predetermined locations can also be evenly distributed on the periphery of the frame, with respect to each other.

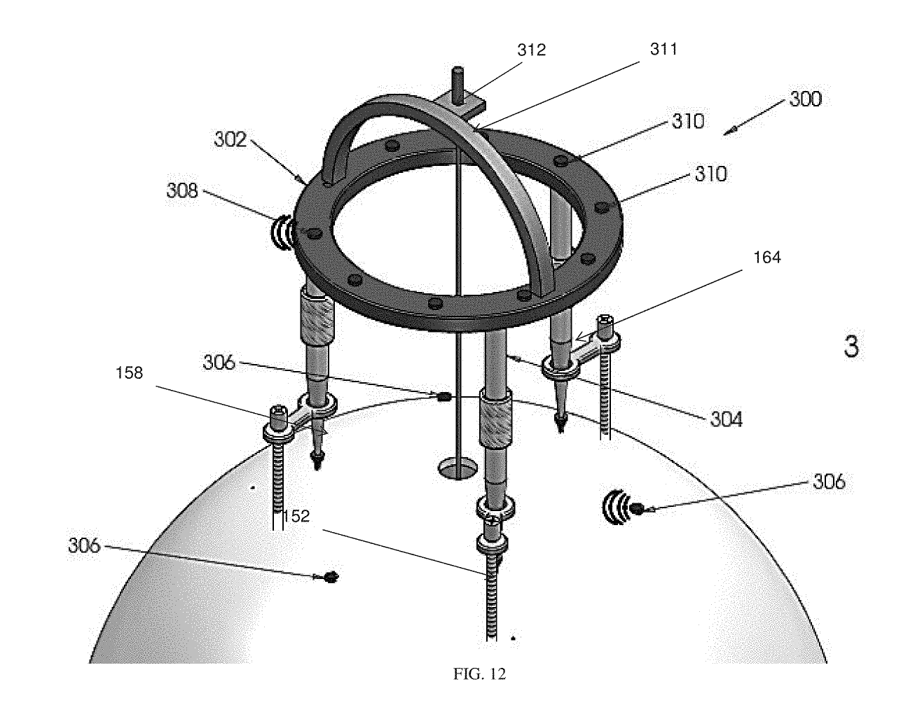

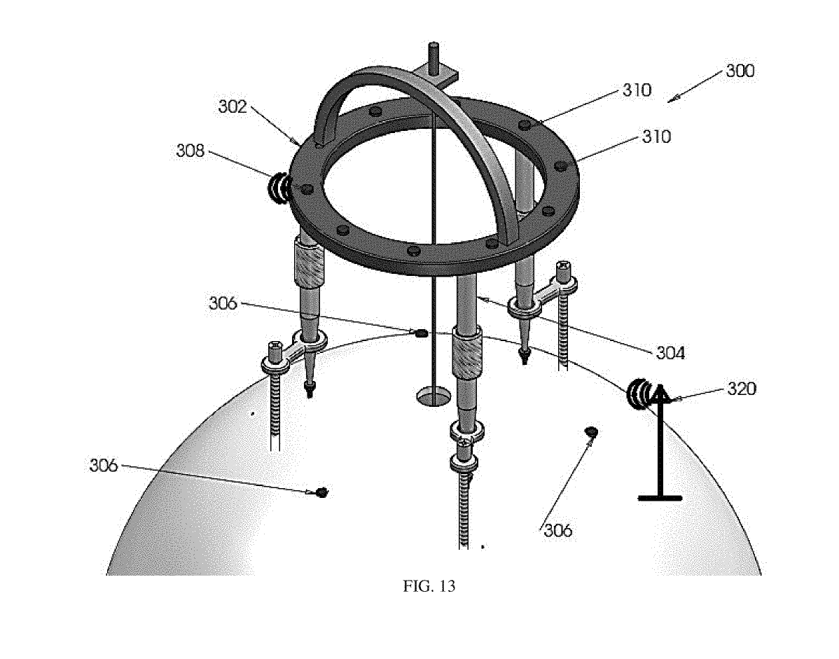

Also, the stereotactic surgical frame further comprises: a circular platform ring having an upper surface and a lower surface with a sensor array radially distributed on the upper surface; a support arc spanning the circular platform ring circumference (in other words, the arc having a chord that is equal to the diameter of the circular (flat) platform ring, the arc rising from the upper surface of the (flat) circular platform ring; and a tab, extending horizontally from the support arc's apogee (its highest point relative to the flat circular ring plane), parallel with the circular platform ring, the tab defining an aperture therein, configured to accommodate and engage a surgical tool (see e.g., FIGS. 12-13), while still allowing the surgical tool (e.g., an electrode) some 360 degrees maneuverability such that its tip can move at least 1 mm in any direction.

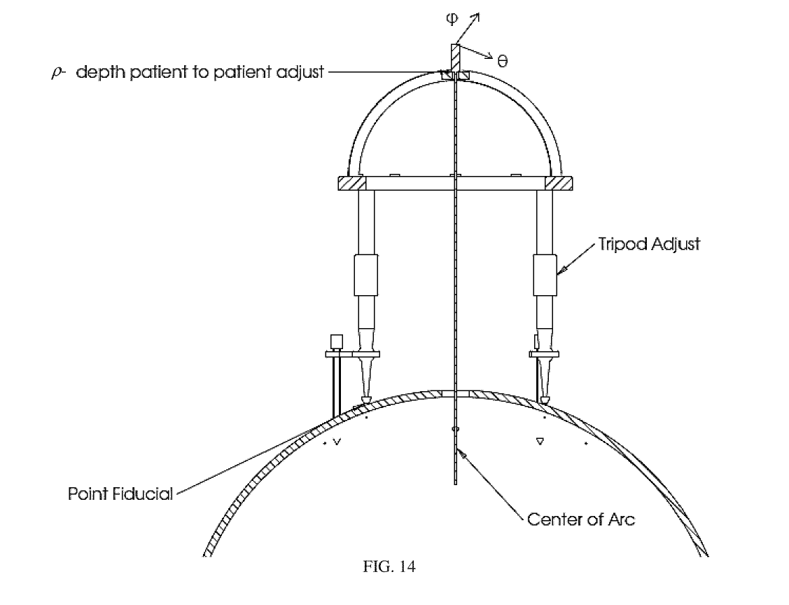

Moreover, provided herein is a stereotactic surgical frame for facilitating insertion of a surgical tool (e.g., a DBS electrode) into a surgical site within a patient body, comprising: a convex domed portion (e.g., a bowl turned upside down), having an open circumferential basal lip (e.g., the lip of the bowl now facing the patient), the convex dome defining an aperture at its apex (the top of the bowl), the aperture configured to receive and engage (for example, via friction engagement) a spherical cap portion (the cap can complete the portion of the sphere defined by the bowl). The spherical cap portion, can be movable with respect to the aperture of the convex domed portion and concentric therewith; and at least three support rods or pods as described hereinabove, having an upper end operably coupled to the open circumferential basal lip. Additionally, the spherical cap portion is configured for adjustment of radial set up parameters (in other words, have markings for spherical coordinate system adjustment, e.g., distance ρfrom the apogee of the dome, and two angles ϕ and θ each at 90 degrees to the other) of said surgical tool relative said stereotactic surgical frame (see e.g., FIG. 14). As indicated, the convex domed portion and the spherical cap portion form a substantial portion of a hemisphere.

The system can be configured such that the distance between a pole of the substantial portion of a hemisphere created by the convex domed portion and the cap or the tip of any knob, turn screw or handle residing on the pole; and the targeted surgical site (e.g., the tip of the surgical tool) can be configured to be proportional to the radius (D₁) defined by said hemisphere. Accordingly, the targeted surgical site and an entrance point of said surgical tool into the spherical cap portion are located on the circumference of imaginary sphere created by the completion of the substantial portion of the hemisphere formed by the dome and the cap.

Further provided is a kit comprising the components provided hereinabove, capable of being assembled to form the systems described herein.

A more complete understanding of the components, processes, assemblies, and devices disclosed herein can be obtained by reference to the accompanying drawings. These figures (also referred to herein as “FIG.”) are merely schematic representations (e.g., illustrations) based on convenience and the ease of demonstrating the present disclosure, and are, therefore, not intended to indicate relative size and dimensions of the devices or components thereof and/or to define or limit the scope of the exemplary embodiments. Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

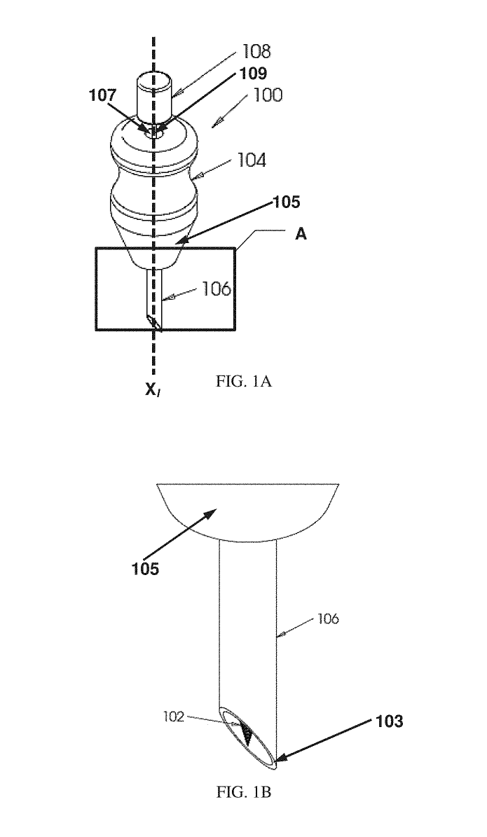

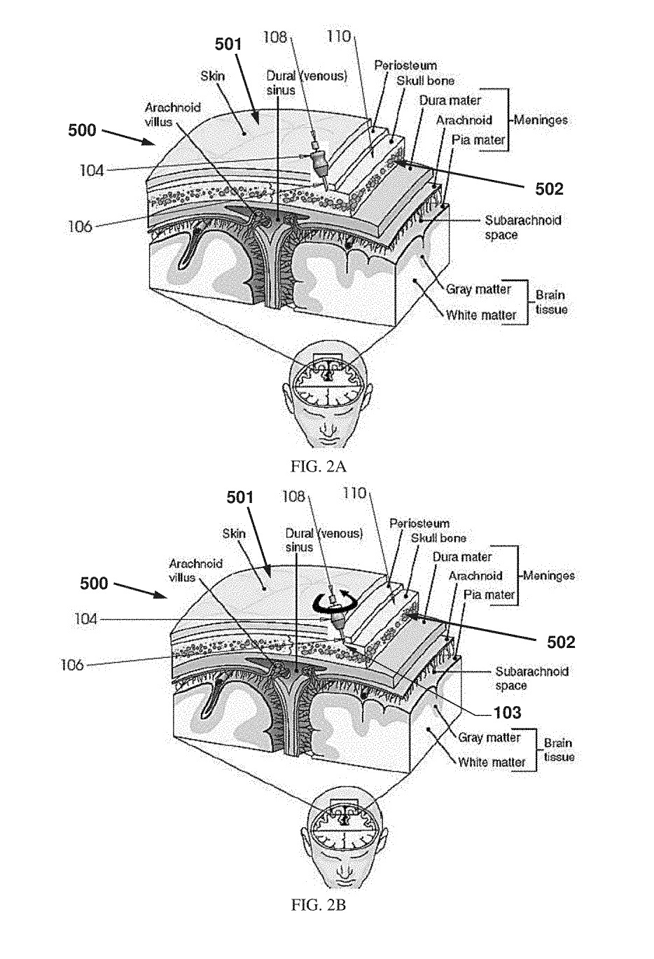

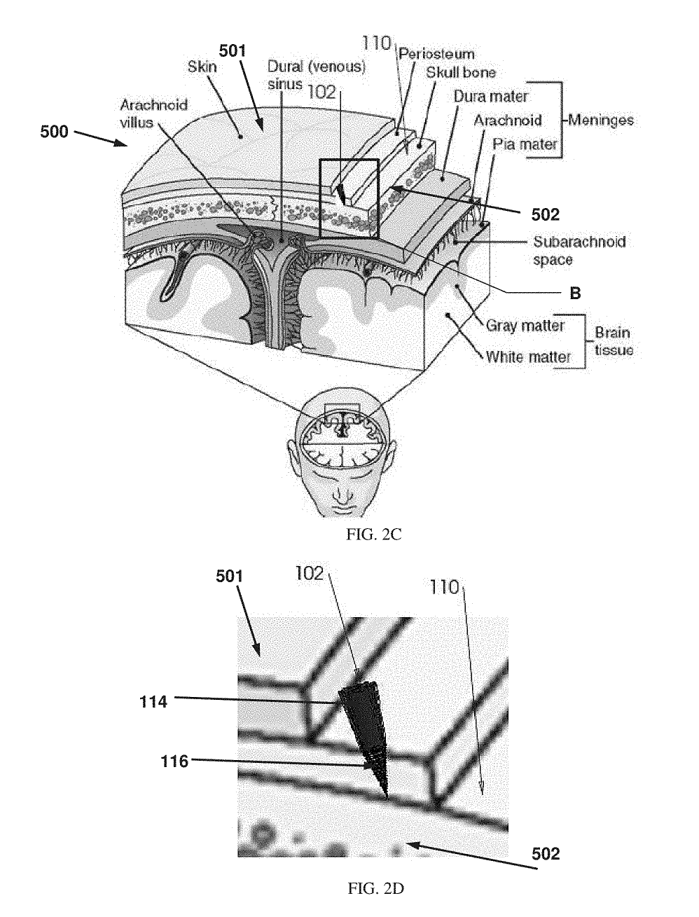

Turning now to FIGS. 1A-1B, illustrating a device for imbedding of fiducials in a body organ of a patient, constructed and operative in accordance with an embodiment of the present invention and to FIGS. 2A-2D, which are simplified illustrations showing various stages of imbedding of a fiducial into a skull of a patient using the device shown in e.g., FIG. 1A.

It can be seen in FIGS. 1A-2C, that miniature fiducials can be fabricated so that they can be imbedded in the patients' skull using a needle or sharpened cannula, thus not requiring incision or anesthetic. These kind of fiducials can be imbedded in the skull of a patient by a non-surgeon, thus substantially simplifying the procedure.

As illustrated in FIGS. 1A, 1B, fiducial threading device 100 can comprise housing 104 having a longitudinal axis Xi with an apical end and a basal end, with bore 107 extending axially; cannula (or needle) 106, operably coupled to basal end 105 of housing 104, with rod 109, having an apical end coupled to knob 108 and a basal end of rod 109 coupled to fiducial 102 (See e.g, FIG. 1B) and fiducial 102, having an apical end configured to releasably engage the basal end of rod 109, and a basal end of fiducial 102 configured to rotatably penetrate and engage a bone tissue of a subject. It is appreciated that in an alternative embodiment, the (plunger) rod 109 may be axially advanced in order to imbed fiducial 102 in bone 502 (see e.g., FIG. 2A), such as by hammering and that the basal end of fiducial 102 can be configured to have a surface to facilitate such imbedding.

Turning to FIGS. 2A-2D, as seen in FIG. 2A fiducial 102 can be attached to needle or cannula 106 and the user can advance cannula 106 such that it penetrates the skin of a patient until reaching bone 502. As seen in FIG. 2B rotating of device 100 can cause threading of fiducial 102 into a fixed position within bone 502. Once fiducial 102 is imbedded in bone 502, further threading of fiducial 102 using rotation of (plunger) rod 109 by knob 108, or axial advancement thereof can cause excessive force to be applied on device 100 and thus fiducial 102 can either breaks off from rod 109, or rotation in the opposite direction, release the rod.

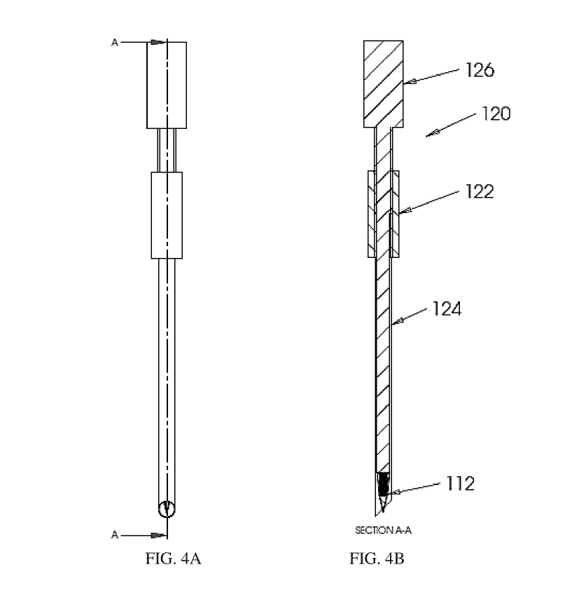

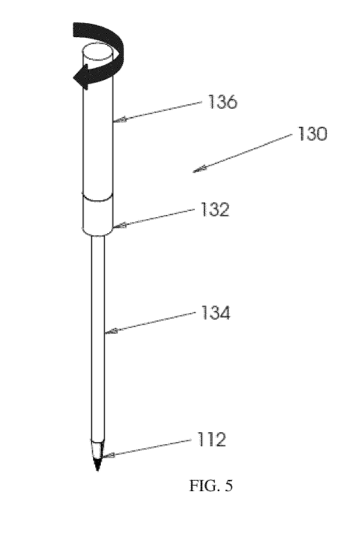

In an embodiment, cannula 106 can be used to perform an opening within a tissue of a patient's body and fiducial 102 can be part of the cannula 106 (see e.g., FIGS. 4, 5). Once excessive force is applied on needle or cannula 106, it can break at a failure point arranged thereon and fiducial 102 can be released and be imbedded in bone 502.

In an alternative embodiment (e.g., FIG. 5), fiducial 102 can be disposed within the needle (interchangeable with cannula) 106 but does not constitute a part thereof, thus once the needle 106 penetrates the tissue of a patient's body, the (plunger) rod 109 can be further advanced or rotated by knob 108, such that fiducial 102 can break off from device 100 such that it can be released from said needle 106 and imbed within bone 502 of the patient. Alternatively, fiducial 102 may be released from device 100 upon exertion of excessive and sudden torque on (plunger) rod 109.

Additionally, it is noted that a mechanism can be provided for an audible verification of placement, such as a click sound in device 100 so that when a certain torque level is obtained, a sound can be heard indicating to the user that fiducial 102 is firmly imbedded within bone 502.

As it is seen in FIG. 2C, once the fiducial 102 is firmly imbedded within bone 502, device 100 is retracted and fiducial 102 remains subcutaneously imbedded within bone 502.

Turning now to FIGS. 3A-3E, illustrating a fiducial assembly, constructed and operative in accordance with another embodiment. As seen in FIGS. 3A-3E, fiducial 112 can be comprised of a generally cylindrical body 114 and a generally conical outwardly threaded portion 116 connected to cylindrical body 114. It is also seen that there are several longitudinal slits 118 axially positioned along cylindrical body 114 in order to provide for relative resiliency of cylindrical body 114. Fiducial 112 is seen in a closed position in FIGS. 3A-3B where cylindrical body 114 has an even circular cross-section of a first diameter. A generally cylindrical coupling member 110 is seen in FIG. 3C, which can be internally threaded 111. Fiducial 112 is seen in an open position in FIGS. 3D-3E. As seen, insertion of cylindrical coupling member 110 into the interior of cylindrical body 114 causes deflection thereof due to the resiliency provided by slits 118 and thus cylindrical housing 114 can obtain a conical shape and have a circular cross-section of a second diameter, which is generally greater than the first diameter (see e.g., FIG. 3E).

It is noted that the fiducial 112 is generally of a small diameter, such as for example 1 mm, thus in accordance with an embodiment of the present invention, during subcutaneous insertion of fiducial 112, the head of the fiducial 112 enlarges due to deflection of the housing 114 and provides support for a frame pod and an ability for the imaging software to automatically detect the center of the fiducial 112, as will be described in detail hereinbelow.

Reference is now made to FIGS. 4A-4B, illustrating a device for imbedding fiducial shown in FIGS. 3A-3E into a body of a patient, wherein the fiducial is positioned within a needle. As illustrated, disposable device 120 is shown which can be preloaded with miniature fiducials 112. Device 120 can be formed of housing portion 122, needle 124 which can be attached to or integrally formed with housing portion 122 and protrudes therefrom; and an actuator 126, which can be movably coupled with respect to housing portion 122, for example rotatbly. A nurse can penetrate the skin of the patient using device 120 until needle 124 reaches bone 502 and then actuator 126 can be configured to be rotated and subsequently advance cylindrical coupling member 120 into fiducial 112. This rotation of actuator 126 can cause deflection of fiducial 112, as described hereinabove, fiducial 112 can then be, for example, screwed into its desired location and released from device 120.

Reference is now made to FIG. 5, illustrating another embodiment of a device for embedding of a fiducial shown in FIGS. 3A-3E into a body of a patient, wherein the fiducial forms part of the needle. As illustrated in FIG. 5, disposable device 130 is shown with miniature fiducials 112. As illustrated, device 130 can be formed of housing portion 132, needle 134 which can be attached to or integrally formed with housing portion 132 and protrudes therefrom and actuator 136 which can be movably coupled with respect to housing portion 132,e.g., rotatbly coupled. As illustrated, nurse can penetrate the skin of the patient using device 130 until needle 134 reaches bone 502 and then the actuator 136 can be configured to be rotated and subsequently advance cylindrical coupling member 120 into fiducial 112. This rotation of actuator 136 can cause deflection of fiducial 112, as described hereinabove, fiducial 112 can then be screwed into its desired location and released from the device 130.

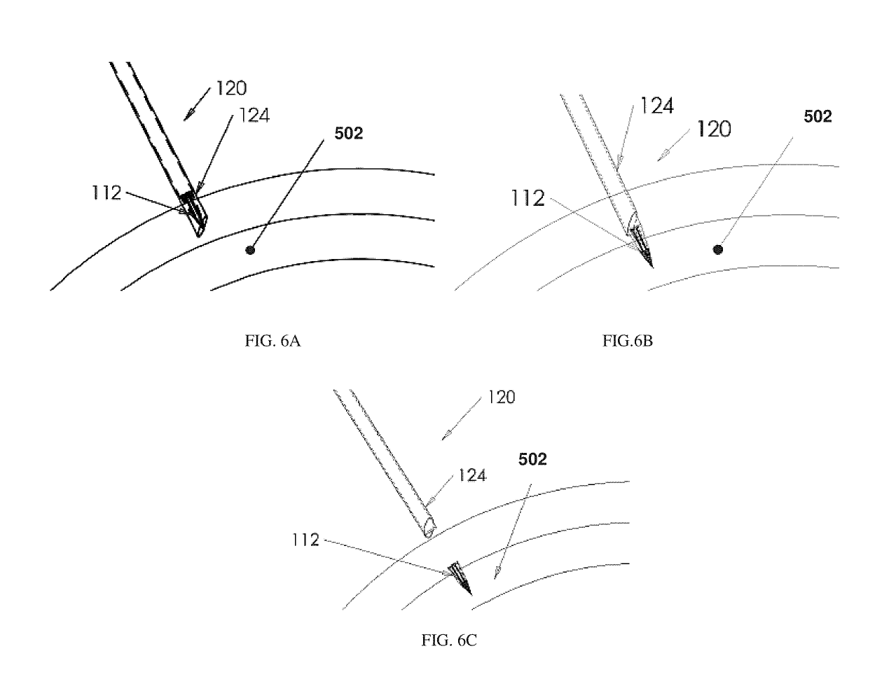

Reference is now made to FIGS. 6A-6C, illustrating various stages of insertion of fiducial 112 into a skull of a patient using device 120 shown in FIG. 4. As illustrated in FIG. 6A, fiducial 112can be positioned within needle 124 and user advances needle 124 such that it penetrates the skin of a patient until reaching bone 502. As seen in FIG. 6B, twisting of device 120 can cause threading of fiducial 112 into a fixed position within bone 502. Once fiducial 112 is imbedded within bone 502, further threading of fiducial 112 using rotation of actuator 126 causes deflection of fiducial 112 and its release from the device 120.

As illustrated in FIG. 6C, once fiducial 112 is firmly imbedded within bone 502, device 120 can be retracted and fiducial 112 remains subcutaneously imbedded within bone 502.

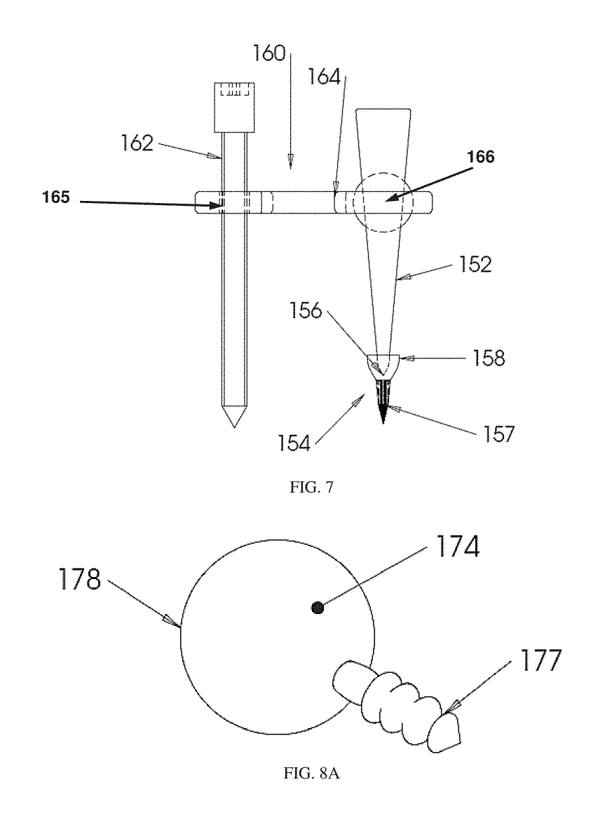

FIG. 7, is a simplified illustration of an embodiment of a stereotactic frame pod assembly, showing an example of a point to point mating configuration between frame pod 152 and fiducial 154. During surgery, edge 156 of frame pod 152 can be attached to the previously imbedded fiducial 154, as was described in detail hereinabove, and frame pod 152 can be tightened to bone 502 (see e.g., FIG. 9C), for example to the skull. As illustrated, edge 156 of frame pod 152 can be formed as a downwardly tapered cone having a basal end or lower end.

As illustrated fiducial 154 can further have screw 157 and an upper concave, cup shaped surface 158 which is exposed and the end point of edge 156 of frame pod 152 can be configured to engage with the cup shaped surface 158 of fiducial 154 in a point to point mating manner, i.e. the end point of edge 156 of frame pod 152 engages the center of the cup shaped surface 158. It is appreciated that point to point engagement is different than axis to axis engagement in that axis to axis engagement requires withstanding accurate production tolerances in order to enable mating and additionally, the patient may inadvertently displace the mating. Whereas in point to point or ball to bell engagements, there is no such requirement in withstanding accurate production tolerances and the patient cannot displace the mating.

Once frame pod 152 is mated point to point with the fiducial 154, the coordinate of the end point of edge 156 of frame pod 152 and the coordinate of the center of cup shaped surface 158 of fiducial 154 are the same or located at a known geometrical relation (Cartesian or spherical coordinates e.g.,) with respect to each other. Subsequently, the frame can be co-registered with the imaging coordinate system or vice versa, thus co-registering the imaging coordinate system with the coordinates of the fiducials 154. As illustrated in FIG. 7, assembly 160 is seen in FIG. 7 and can include screw 162 and transverse coupling member 164. Once the frame pod 152 is mated with the fiducial 154 in a point to point manner as described hereinabove, this mating is positioned by the assembly 160, such that the screw 162 is threaded in to the bone, for example into the skull.

The screw 162 is connected to transverse coupling member 164, in accordance with an embodiment of the present invention, it is a ball joint. This connection of the screw 162 with transverse coupling member 164 provides for keeping the mating between the frame pod 152 and the fiducial 154 in fixed position. It is appreciated that any other suitable kind of transverse coupling member 164 may be used in this positioning assembly 160 instead of the ball joint.

Reference is now made to FIGS. 8A-8D, illustrating an engagement between a surgical frame and a fiducial (8A), with, FIG. 8D is a Z-X sectional illustration of FIG. 8C. As illustrated, frame pod 172 and a fiducial 174 during the surgery, lower end portion 176 of the frame pod 172 can be attached to the previously imbedded fiducial 174, as was described in detail hereinabove, and the frame pod 172 is tightened to bone 502, for example to the skull. As illustrated in FIG. 8B, the lower end portion 176 of the frame pod 172 can be formed as a generally deformable hollow ball receiving bell-shaped element having an opening at its lower end. As illustrated in FIG. 8D, engagement between the frame pod 172 and the fiducial 174, allows center to center attachment of the frame pod 172 and the fiducial 174, rather than axis to axis attachment. Furher, as seen in FIG. 8C, fiducial 174 can have screw 177 and an upper generally spherical portion 178 which is exposed and the lower end portion 176 of frame pod 172 is configured to mate with the spherical portion 178 of fiducial 174 in a center to center mating manner, i.e. the center of lower end portion 176 of frame pod 172 is aligned with the center of spherical portion 178 of fiducial 174.

It can be appreciated that center to center mating is different than axis to axis mating in that axis to axis mating requires withstanding accurate production tolerances in order to enable mating and additionally, the patient may inadvertently displace the mating. Whereas in center to center mating, there is no requirement in withstanding accurate production tolerances and the patient cannot displace the mating. Accordingly and as illustrated, once the lower end portion 176 of frame pod 172 engages the spherical portion 178 of fiducial 174, the lower end portion 176 of frame pod 172 is being deformed, thus enlarging its opening, in order to accommodate the spherical portion 178 and once full engagement is obtained, a click sound can be provided for verification of mating between the frame pod 172 and the fiducial 174.

Once the frame pod 172 is mated center to center with the fiducial 174, the coordinate of the center of the lower end portion 176 of frame pod 172 and the coordinate of the center of the spherical portion 178 of fiducial 174 are aligned or located at a known geometrical relation with respect to each other and are co-registered with any imaging taken using the imbedded fiducial(s). Subsequently, the frame can be co-registered with the imaging coordinate system or vice versa, thus co-registering the imaging coordinate system with the coordinates of the fiducials 174. Similar engagement is illustrated in FIGS. 9A-9C, besides the fact that in the embodiment shown in FIGS. 9A-9C a positioning mechanism 180 is seen added.

Positioning mechanism 180 comprises a screw 182 and a joint 184. Once the frame pod 172 is mated with the fiducial 174 in a center to center manner as described hereinabove, this mating is fixated by the fixating mechanism 180, such that the screw 177 of the fiducial 174 is threaded in to the bone, for example into the skull. The screw 182 is connected to a joint 184, and is rotated to position the joint 184 in order to lock the frame pod 172 relative to the fiducial 174. This connection of the screw 182 with the joint 184 provides for keeping the mating between the frame pod 172 and the fiducial 174 in fixed position. It is appreciated that any other suitable kind of joint 184 may be used in this positioning mechanism 180 instead of joint 184.

Reference is now made to FIGS. 10A-10E, illustrating an engagement configuration embodiment between a surgical frame and a fiducial. FIG. 10B is a Z-X sectional illustration taken along lines C-C in FIG. 10A and FIG. 10E is a Z-X sectional illustration taken along lines D-D in FIG. 10D. FIGS. 10A-10E having a frame pod 192 and a fiducial 194. During the surgery, lower end portion 196 of the frame pod 192 is preferably attached to the previously implanted fiducial 194, as was described in detail hereinabove. As illustrated, the lower end portion 196 of the frame pod 192 is formed generally as a spherical portion. The engagement between frame pod 192 and fiducial 194 allows center to center attachment of the frame pod 192 and the fiducial 194, rather than axis to axis attachment. As seen in FIG. 10B, the imbedded fiducial 194 can have a screw 197 and an upper generally hollow bell-shaped portion 198 having a ball receiving opening at its upper end, which is exposed. The lower end portion 196 of frame pod 192 is configured to mate with the bell-shaped portion 198 of fiducial 194 in a center to center mating manner, i.e. the center of spherical lower end portion 196 of frame pod 192 is aligned with the center of bell-shaped portion 198 of fiducial 194. Once the spherical lower end portion 196 of frame pod 192 engages the bell-shaped portion 198 of fiducial 194, the bell-shaped portion 198 of fiducial 194 is being deformed, thus enlarging its opening, in order to accommodate the spherical lower end portion 196 of the frame pod 192 and once full engagement is obtained, a click sound is provided for verification of mating between the frame pod 192 and the fiducial 194.

In an embodiment, bell-shaped portion 198 of the fiducial 194 is preferably slotted, thus providing some resiliency for receiving the spherical lower end portion 196 of the frame pod 192 therein. Alternatively, the fiducial 194 can be formed of a relatively resilient material to provide for the same resiliency requirement. Once the frame pod 192 is mated center to center with the fiducial 194, the coordinate of the center of the spherical lower end portion 196 of frame pod 192 and the coordinate of the center of the bell-shaped portion 198 of fiducial 194 are aligned or located at a known geometrical relation with respect to each other. Subsequently, the frame can be co-registered with the imaging coordinate system or vice versa, thus co-registering the imaging coordinate system with the coordinates of the fiducials 194.

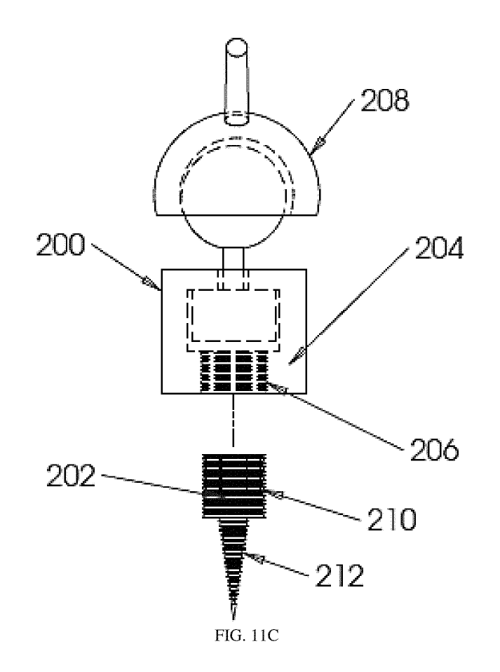

Turning now to FIGS. 11A-11C, which are simplified pictorial illustrations of an engagement between a surgical frame and a fiducial, constructed and operative in accordance with a fifth embodiment of the present invention, FIG. 11C is a Z-X sectional illustration taken along lines E-E in FIG. 11B. As illustrated, frame pod 200 is generally comprised of a hollow housing portion 204 having a lower inwardly threaded end portion 206 and a ball joint structure 208, which is coupled to the housing portion 204, with fiducial 202 having an upper externally threaded portion 210 and a generally conical screw 212 protruding downwardly therefrom. During the surgery, lower end portion 206 of the frame pod 200 can be attached by threading to the upper portion 210 of the previously implanted fiducial 202. Further, the engagement between frame pod 200 and fiducial 202 allows center to center attachment of the frame pod 200 and the fiducial 202, rather than axis to axis attachment. Once the frame pod 200 is mated center to center with the fiducial 202, the coordinate of the center of the fiducial upper portion 210 and the coordinate of the center of the lower end portion 206 are aligned or located at a known geometrical relation with respect to each other. Subsequently, the frame can be co-registered with the imaging coordinate system or vice versa, thus co-registering the imaging coordinate system with the coordinates of the fiducials 202.

Turning now to FIGS. 12 and 13, illustrating stereotactic frame in a co-registration process with an imaging coordinate system, where stereotactic frame 300 which can enable automatic co-registration thereof with an imaging coordinate system. In contrast to the engagements between frame pods and fiducials which are illustrated in FIGS. 7-11C, providing mechanical connection between the frame and the fiducials, FIG. 12 illustrates an alternative embodiment including a non-mechanical mating between the frame and the fiducials and thus enables to co-register the frame with the imaging coordinate system while the frame pods are not mechanically attached to the implanted fiducials. As shown frame 300 is setup during the surgery in order to accurately direct a surgical tool towards the targeted surgical site. The surgeon can adjust the setup of the frame 300 during the surgery. Frame 300 can be configured to automatically detect the coordinates of imbedded fiducials, as described in detail hereinabove, and thus enabling co-registration between the frame 300 and the imaging coordinate system.

As illustrated, frame 300 includes an upper portion 302 and a plurality of frame pods 304. It is additionally seen that a plurality of fiducials 306 are implanted within the skull, preferably by methods which are described in detail hereinabove. The fiducials 306 can be either entirely imbedded within the bone of a patient or partially exposed above the skin of the patient. Additionally, the stereotactic surgical frame 300 can comprise: a circular platform ring 302 having an upper surface and a lower surface with a sensor array 308, 310 (e.g., transceivers) radially distributed on the upper surface; a support arc 311 spanning the circular platform ring 302 circumference rising from the upper surface of the circular platform ring 302; and a tab 312, extending horizontally from the support arc's 311 apogee, parallel with the circular platform ring 302, the tab 312 defining an aperture therein, configured to accommodate and engage a surgical tool. Transceivers 308, 310 can be configured for transmitting a signal to the fiducials 306, such as an optical, RF, infra-red, magnetic or ultrasonic signal for example. Once the signal reaches the fiducials 306, an echo is returned and received by the transceivers 310.

It is noted that in prior devices, an external tool was required in order to co-register the frame and the fiducials. In accordance with an embodiment of the present invention, this external tool is obviated, since the transmitters and the detectors are formed as an integral part of frame 300.

Once the transceivers 310 have identified the locations of fiducials 306, the exact position of each fiducial 306 is calculated relative to the coordinate system of frame 300 and then the frame 300 can be automatically co-registered with the imaging coordinate system. The frame 300 is generally set-up such that the central trajectory thereof is aligned with the targeted surgical site.

Turning now to FIG. 13, illustrating frame 300 that can be fixed at an arbitrary location of the skull of a patient and a wireless extension tool 320, which is associated with the frame having transmitters 308 and detectors 310 as an integral part thereof may be employed. This wireless extension tool 320 is displaced over the skull in order to identify the location of fiducials 306 by transferring signals from the transmitter and receiving an echo from the fiducials 306. The wireless extension tool 320 is configured for registering the locations of the fiducials 306 and transmit these locations to detectors 310 located on frame 300. Following combination of all readings registered by this wireless extension tool 320, geometrical relation between the frame 300 and the fiducials 306 can be established and thus the frame 300 is co-registered with respect to the imaging coordinate system. It is appreciated that the extension tool 320 can alternatively be wired. Thus, frame 300 can be placed in an arbitrary location on the skull of a patient and is configured to automatically identify the location of fiducials 306 using a detection mechanism based on RF, optical, infra-red, magnetic or ultrasonic signals, as described in detail hereinabove. This detection allows for co-registering the coordinates of the frame and the coordinates of the imaging coordinate system, while obviating an external tool for detection of the fiducial locations.

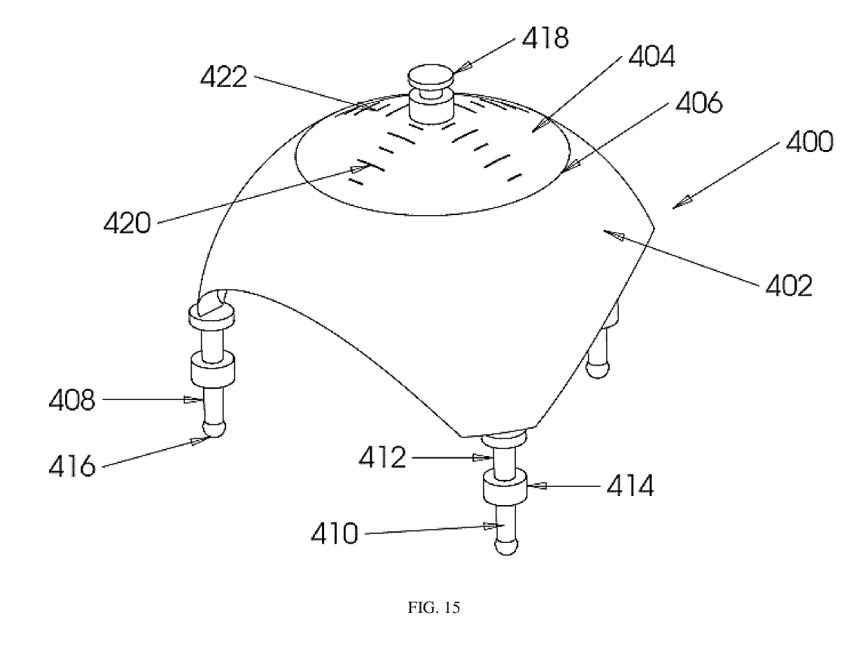

Turning now to FIGS. 14 and 15, illustrating a frame defining a spherical coordinate system. As illustrated, frame 400 is seen in FIG. 15. In an embodiment, stereotactic surgical frame 400 for facilitating insertion of a surgical tool into a surgical site within a patient body, comprising: a convex domed portion 402 having an open circumferential basal lip (415 not shown), the convex dome 402 defining an aperture 406 at its apex, the aperture 406 configured to receive and engage a spherical cap portion 404; the spherical cap portion 404, being movably coupled to the aperture 406 of the convex domed portion 402 and concentric therewith; and at least three support rods, having an upper end 412 operably coupled to the open circumferential basal lip. Spherical cap portion 404 is moveable with respect to convex domed portion 402 in order to allow adjustment of radial set up parameters “α” and “β”.

The stereotactic frame 400 can include the following setup parameters: each of the frame pods can be axially displaced, along (telescopic) members e.g., 410, 414, 412; the upper portion of the stereotactic frame 400 can be axially displaced to adjust the depth adaptor as indicated by arrow designated by “z” and the upper portion of the frame can be radially displaced in several different directions as indicated by arrows designated by “α” and “β”. It is appreciated that more than three frame pods may be utilized in order to improve accuracy. It is noted that while calculating frame setup parameters, a software is configured for utilizing an optimization algorithm in order to provide for “α” and “β” initial value which is generally around zero in order to maximize the movement range allowed for the surgeon in order to further adjust the values of “α” and “β” during the surgery.

As illustrated, at least three frame pods 408 are coupled to the main convex domed portion 402, although it is appreciated that more frame pods 408 can be utilized. Frame pods 408 can be formed of two cylindrical elements 410 and 412 which are relatively moveable with respect to each other in a telescopic manner. A knob 414 is formed on each frame pod 408 in order to allow adjustment of the length thereof. It is appreciated that any other suitable mechanism permitting length adjustment of the frame pod 408 may be also utilized. Each frame pod 408 has an upper end which is connected to the main bottom frame portion 402 and an opposite lower end 416, which is configured for point to point mating with a fiducial as described in detail hereinabove. For example, knob 418 can be formed generally at the center of moveable upper frame portion 404 configured to adjust the “z” parameter (or p in spherical coordinate system), thus adjusting the total distance from the tip of the surgical tool to the targeted surgical site. Likewise, marking scales 420 and 422 can be denoted on the outer surface of the moveable spherical cap portion 404 in order to identify adjustment made to radial parameters “α” and “β” (or θ and ϕ in spherical coordinate system). It is appreciated that the marking scales, such as 420 and 422 could alternatively be positioned on convex domed portion 402. A locking mechanism is provided in order to position the spherical cap portion 404 relative the convex domed portion 402 once the adjustment is completed.

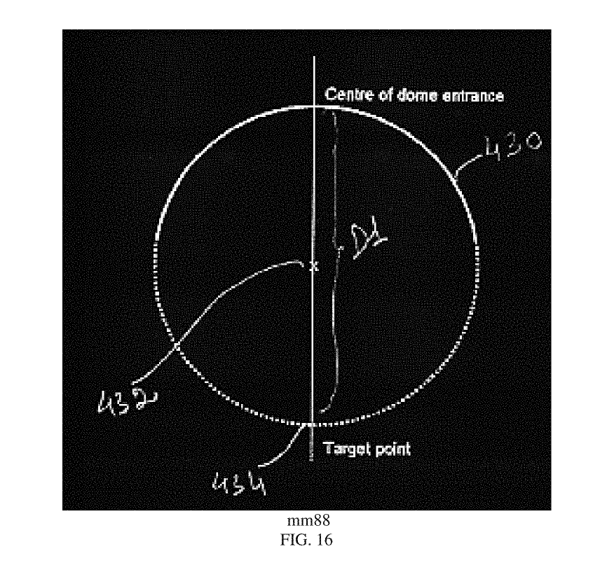

Reference is further made to FIGS. 16 & 17, which are simplified schematic illustrations of targeting a surgical tool using the hemispherical stereotactic frame 400 shown in FIG. 15. As illustrated, the shape of the spherical cap portion 404 is part of an imaginary sphere having a diameter D₁, designated by reference numeral 430 and having a center point 432. As illustrated, the distance between the upper point of the imaginary sphere 430 and the targeted surgical site, designated by reference numeral 434, is equal to twice the radius of the virtual sphere, thus the center point 432 of the imaginary sphere 430 is half way to the targeted surgical site 434. Alternatively, it is appreciated that any other ratio may be utilized, such as for example, displacement of the surgical tool by 1 mm resulting in displacement of the targeted surgical site by 2 mm or 0.5 mm or alike. The displacement of the targeted surgical site corresponds to the displacement of the surgical tool in accordance with the chosen ratio.

Therefore, the targeted surgical site 434 can be adjusted, for example, by a radial shifting of a first value in a first direction, by shifting spherical cap portion 404 by the same first value in a second direction, which is opposite to the first direction. This correlation between the shifting of spherical cap portion 404 and the adjustment of targeted surgical site 434 occurs due to the fact that both the targeted surgical site 434 and the entrance point of the surgical tool to the stereotactic frame 400 are located on the circumference of imaginary sphere 430.

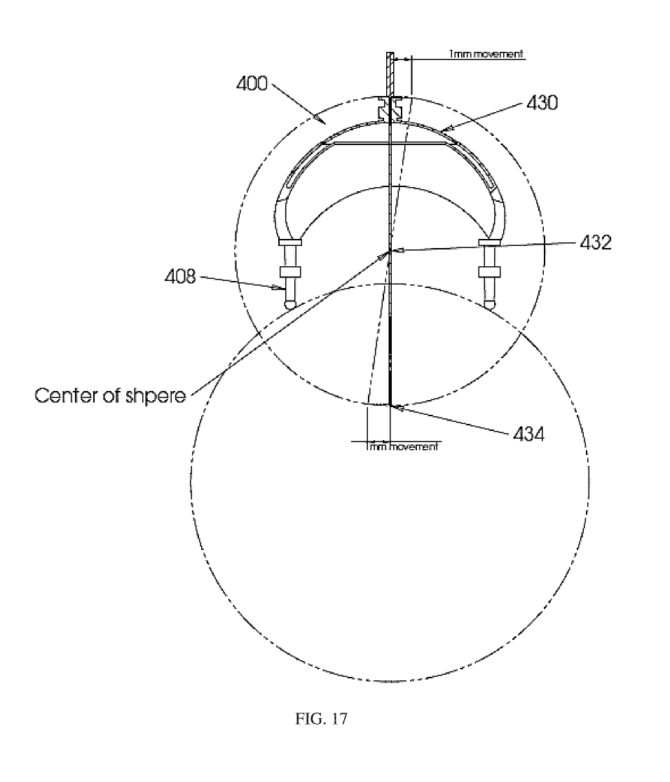

An example of targeted surgical site adjustment by the spherical frame 400 is seen in FIG. 17. Adjustment of 1 mm of the targeted surgical site in a clockwise direction can be performed by adjustment of the entrance point within spherical cap portion 404 by 1 mm in a counter clockwise direction. This feature allows to visualize adjustments of the targeted surgical site during surgery without referring to any further calculations. It is appreciated that any other ratio may be utilized, such that an adjustment of 1 mm of the targeted surgical site in a clockwise direction can be performed by adjustment of the entrance point within the moveable top frame portion 404 by 2 mm in a counter clockwise direction or by 0.5 mm in a counter clockwise direction or alike. As illustrated, the movement range of radial adjustment of the surgical tool is maximized while using the spherical stereotactic frame 400, since the spherical shape of the frame 400 allows for closer disposition of the center point 432 to the entry point to the skull and thus the range of radial maneuvering of the surgical tool is maximized.

It is additionally noted that in accordance with an additional embodiment of the present invention, an electrode with a driving shaft may be utilized.

The electrode preferably has a proximal end and a distal end. There are a plurality of contacts at the distal end of the electrode. The contacts may be formed in any suitable configuration. The proximal end of the electrode is configured to connect using a single connection to an opposite connector (not shown) which supplies driving shaft and electrical connection to the system. The electrode preferably employs all required mechanisms for implanting a DBS lead, take biopsies or perform electrode tip protection.

Detailed embodiments of the present technology are disclosed herein; however, it is to be understood that the disclosed embodiments are merely exemplary, which can be embodied in various forms. Therefore, specific structural and functional details disclosed herein are not to be interpreted as limiting but merely as a basis for the claims and as a representative basis for teaching one skilled in the art to variously employ the present technology in virtually any appropriately detailed structure. Further, the terms and phrases used herein are not intended to be limiting but rather to provide an understandable and enabling description.

The terms “first,” “second,” and the like, herein do not denote any order, quantity, or importance, but rather are used to denote one element from another. The terms “a”, “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the fiducial(s) includes one or more fiducial). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

In addition, for the purposes of the present disclosure, directional or positional terms such as “top”, “bottom”, “upper,” “lower,” “side,” “front,” “frontal,” “forward,” “rear,” “rearward,” “back,” “trailing,” “above,” “below,” “left,” “right,” “horizontal,” “vertical,” “upward,” “downward,” “outer,” “inner,” “exterior,” “interior,” “intermediate,” etc., are merely used for convenience in describing the various embodiments of the present disclosure.

One or more components may be referred to herein as “configured to,” “configured by,” “configurable to,” “operable/operative to,” “adapted/adaptable,” “able to,” “conformable/conformed to,” etc. The terms (e.g. “configured to”) can generally encompass active-state components and/or inactive-state components and/or standby-state components, unless context requires otherwise.

While in the foregoing specification the stereotactic systems and methods of co-registration of stereotactic frames with imbedded fiducial markers have been described in relation to certain preferred embodiments, and many details are set forth for purpose of illustration, it will be apparent to those skilled in the art that the disclosure can be susceptible to additional embodiments and that certain of the details described in this specification and as are more fully delineated in the following claims can be varied considerably without departing from the basic principles of this invention.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method for co-registering a stereotactic frame and a fiducial comprising:
connecting a stereotactic frame defining a surface of a sphere patch and comprising a movable holder configured to hold a surgical tool and to move along said surface, to an organ;
co-registering said movable holder and/or said frame to one or more fiducials imbedded in said organ in know locations.

2. A method according to claim 1, comprising fixing said movable holder at a selected position on said sphere surface.

3. A method according to claim 2, comprising inserting said a surgical tool into an aperture of said movable holder.

4. A method according to claim 1, wherein said co-registering comprises co-registering said movable holder and/or said frame using one or more sensors coupled to the frame, wherein said one or more sensors are in communication with said fiducials.

5. A method according to claim 1, wherein said connecting comprises connecting said frame to said fiducials.

6. A method according to claim 1, wherein said organ comprises a skull.

7. A method according to claim 1, comprising detecting the position of said fiducials using one or more imaging techniques.

8. A method according to claim 1, wherein said surgical tool comprises a deep brain stimulation (DBS) lead.

9. A stereotactic surgical frame for facilitating insertion of a surgical tool into a target surgical site within a patient body, comprising:
a platform defining a curved path;
a movable tool holder comprising an aperture configured to accommodate a surgical tool, and
wherein said tool holder is configured to move along said curved path;
wherein said frame is configured to be co-registered with one or more fiducials imbedded in an organ at know locations.

10. A frame according to claim 9, comprising at least one frame pod having an upper end connected to said platform, wherein said at least one frame pod is connectable to said one or more fiducials.

11. A frame according to claim 9, comprising one or more sensors coupled to said frame, wherein said sensors are configured to communicate with said one or more fiducials.

12. A frame according to claim 9, wherein said platform comprises an arc defining said curved path, and wherein said movable tool holder is configured to move along a surface of said arc.

13. A frame according to claim 9, wherein said platform comprises a patch of a sphere defining said curved path, and wherein said tool holder is configured to move along a surface of said sphere portion.

14. A frame according to claim 9, wherein said organ comprises a skull.

15. A frame according to claim 9, comprising a lock configured to lock said movable tool holder at a selected position along said curved path.

16. A frame according to claim 9, wherein said movable tool holder is configured for adjustment of set up parameters of said surgical tool relative to said frame using spherical coordinates.

17. A frame according to claim 9, wherein said movable tool holder aperture is configured to be positioned together with said targeted surgical site on a circumference of an imaginary sphere.

18. A frame according to claim 17, wherein a distance between said movable tool holder aperture and said targeted surgical site is proportional to a radius defined by said imaginary sphere.

19. A frame according to claim 9, wherein said movable tool holder is configured to allow axial movement of said surgical tool along an axis perpendicular to said curved path.

20. A frame according to claim 9, wherein said surgical tool comprises a deep brain stimulation (DBS) lead.
